(19) United States
(12) Patent Application Publication
(10) Pub. No.: US 2012/0329121 A1
(43) Pub. Date: Dec. 27, 2012

(54) ENHANCING ALGAE GROWTH BY REDUCING COMPETING MICROORGANISMS IN A GROWTH MEDIUM

(76) Inventors: Michael P. Green, Pleasant Hill, CA (US); Scott A. Fraser, Manhattan Beach, CA (US); Nicholas D. Eckelberry, Los Angeles, CA (US); Jose L. Sanchez Pine, Los Angeles, CA (US)

(21) Appl. No.: 13/419,062

(22) Filed: Mar. 13, 2012

Related U.S. Application Data
(60) Provisional application No. 61/454,472, filed on Mar. 18, 2011.

(51) Int. Cl. C12N 13/00

(52) U.S. Cl. 435/173.1

(57) ABSTRACT

A method is described by which a growth medium is exposed to an electric field of sufficient magnitude to kill competing microorganisms and insufficient magnitude to cause flocculation to an algae population.

### RELATED APPLICATIONS

This application claims the benefit of U.S. Provisional Application No. 61/454,472 filed Mar. 18, 2011, entitled ENHANCING ALGAE GROWTH BY REDUCING COMPETING MICROORGANISMS IN A GROWTH MEDIUM, which is incorporated herein by reference in its entirety.

### BACKGROUND

1. Field of the Invention

This application relates generally to methods for enhancing algae growth in a growth medium by reducing foreign species invasion using an electric field.

2. Background

Products which may be derived from biomass, such as the intracellular products of microorganisms, show promise as partial or full substitutes for fossil oil derivatives or other chemicals used in manufacturing products such as, at least in part, pharmaceuticals, cosmetics, nutraceuticals, other food products, industrial products, biofuels, synthetic oils, animal feed, fertilizers and so forth. However, for these substitutes to become viable, methods for both fostering the growth and development of the biomass and obtaining and processing usable bio-based products must be efficient and cost effective in order to be competitive with the refining costs associated with fossil oil derivatives. Current systems and methods used for growing and harvesting bio-based products for use as fossil oil substitutes are laborious and yield low net energy gains, rendering them unfeasible for today's alternative energy demands. Further, such methods can produce a significant carbon footprint, exacerbating global warming and other environmental issues. These methods, when further scaled up, produce an even greater efficiency loss, due to valuable intracellular component degradation, and require greater energy or chemical inputs than what is currently financially and/or environmentally feasible from a commercially viable biomass harvest.

These processes can utilize microalgae biomass feedstocks that can be grown and later harvested for its lipid content. Microalgae are single celled photosynthetic organisms made up of proteins, carbohydrates, fats and nucleic acids in varying proportions. While composition percentage varies among algal species, the lipid content of some of these species can be up to 50% of their overall mass. This lipid content of specific species has attracted attention from industries such as the petro-chemical and pharmaceutical industries as a viable feedstock to replace hydrocarbon sources or food sources. This valuable content, however, also attracts predators, such as ciliates, rotifers and certain bacteria who feed on algae. The mono culturing of specific algae species is designed to attain a certain byproduct with consistency. However, over time, a reactor, photo reactor or even a highly regulated photo-bio-reactor will usually become contaminated by invasive species which might not have the desired characteristics, therefore rendering an uneven harvest or in many cases crashing the whole of the culture, leading to delays due to restocking after complete sterilization. The invasion of the growth tanks by these predators challenges long-term industrial viability of algae-to-product programs. Accordingly, a systems and methods of reducing the population of such predators are herein presented.

### SUMMARY

The present invention relates to a method for inhibiting the growth of competing microorganisms in a growth medium by exposing the growth medium to an electric field sufficient to kill competing microorganisms and insufficient to cause flocculation to an algae population.

Other aspects of the present invention relate to a system for inhibiting the growth of competing microorganisms in a growth medium. The system includes two or more electrodes connected with an electrical power supply. The power supply is configured to apply an electric field across the growth medium, via the two or more electrodes, of sufficient magnitude to kill competing microorganisms and of insufficient magnitude to cause flocculation to an algae population.

Still other aspects of the present invention relate to a system for inhibiting the growth of competing microorganisms in a growth medium. The system includes two or more electrodes connected with an electrical power supply and configured to apply an electric field across a growth medium of sufficient magnitude to kill competing microorganisms and of insufficient magnitude to cause flocculation in an algae population. The system also include one or more sensors configured to sense biofeedback data from the growth medium. A computerized control system of the system is in electronic communication with the one or more sensors. The computerized control system can adjust the parameters of the electric field based on the biofeedback data received from the one or more sensors.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 illustrates a cross-section view of a tank and a set of electrodes, according to a representative embodiment.

FIG. 2 illustrates a cross-section view of a tank and a set of electrodes, according to another representative embodiment.

FIG. 3 illustrates a cross-section view of a bypass pipe and a set of electrodes, according to a representative embodiment.

FIG. 4 illustrates a cross-section, perspective view of set of concentric electrodes, according to a representative embodiment.

FIG. 5 illustrates a cut-away, perspective view of a circular tank having a perimeter wall electrode and a central electrode, according to a representative embodiment.

FIG. 6 illustrates a non-limiting example of various sensor components according to some embodiments of the present invention.

FIG. 7 illustrates a non-limiting example of Supervisory Control and Data Acquisition components according to certain embodiments of the present invention.

FIG. 8 illustrates a non-limiting example of a system according to some embodiments of the present invention.

### DETAILED DESCRIPTION OF THE INVENTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention is grouped into subheadings. The utilization of the subheadings is for convenience of the reader only and is not to be construed as limiting in any sense.

The description may use perspective-based descriptions such as up/down, back/front, left/right and top/bottom. Such descriptions are merely used to facilitate the discussion and are not intended to restrict the application or embodiments of the present invention.

For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).” For the purposes of the present invention, the phrase “(A)B” means “(B) or (AB)”, that is, A is an optional element.

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

The present invention concerns a method and associated apparatus for destroying invading species such as bacteria and rotifers without destroying the cells of a microalgae growth medium. In general, this can be accomplished by exposing microalgae cells, in suspension, to a suitable electric field. This electric field can be strong enough to destroy the foreign invader, but not strong enough to affect the general species grown for product. For example, in particular cases at least 40%, 50%, 60%, 70%, 80%, 90%, 95%, 99%, or 100% of the previously viable cells remain viable following exposure to the electric field. The reduction in the competing microorganisms can enhance the growth the desired algae species grown for product.

The electric field is believed to affect competing microorganism more greatly than algae due to the cell structure of algae. Because microalgae are eukaryotes, microalgae have a protective cell membrane shielding the cells from extremes such as weather, sunlight (ultraviolet radiation) and nutrient deprivation. Prokaryotes and the three major subdivisions of the Rotifera, Seisonacea, Bdelloidea and Monogononta are in the class of invertebrates, and do not have this characteristic protective membrane, and therefore are believed to be more prone to destruction by a targeted electric field.

It will be noted, that the application of an electromagnetic field to a biomass feedstock during growth is contrary to conventional reasoning, which regards electro-manipulation as a method of eradicating, rather than the converse. Conventionally, the utilization of electrolysis and electro-manipulation is intended for killing or damaging invasive species such as algae and bacteria in aqueous environments, such as water treatment facilities, swimming pools, ponds, and the like. Procedures such as electro-flocculation can be relatively inexpensive, effective, non-chemical methods of eradicating such species. Accordingly, exposing the biomass feedstock to an electromagnetic field to aid in growth in aquatic environments is unconventional at best.

Despite its unconventiality, in various embodiments, the application an electromagnetic field can of increase biomass feedstock production. Moreover, the underlying approach of this invention is to stimulate the growth medium, either in part or the whole, through the utilization of an electric field to destroy pathogens which interfere with constant mono-culture growth. Furthermore, this process has been found to be able to be performed on a growth medium multiple times without destroying the cells viability. Thus, this process can minimize the need for chemical treatments, re-incubation or other extreme measures to maintain log growth, and in many cases complete re-inoculation after thorough sterilization. Furthermore, the affected reactors need to immediately be placed under quarantine lest the whole farm be infected by some of these predators.

Embodiments of the present invention can utilizes a device or apparatus in which an electric field is imposed between the anode and cathode across a volume of water containing algal cells, creating an electric current through that medium. The anode and cathode can be electrodes whose configuration creates an effective electric field and/or current within the medium of water and algal cells. Such embodiments are described below.

### Electrode Configurations and Placements

Electrodes for applying an electric field can be configured in many different ways. The electrode may be chosen in conjunction with power supply capabilities, power availability and desired processing capacity.

General examples of electrode configurations can include at least three types: 1) whole container electrodes where all, substantially all, or at least a large fraction of the volume of the container (or tank) simultaneously has an effective electric field when the electrical supply to the electrodes is activated; 2) by-pass or transfer passage electrodes where electrodes are external to the container and are situated in a pipe, tube, or other passage for fluid flow; and 3) submerged isolation electrodes where the electrodes are submerged within the tank but are sufficiently electrically isolated from the bulk medium in the container that at least a large fraction of the current passing between the electrodes follows essentially the shortest path between anode and cathode. With this type of electrode design, the bulk medium in the container is not exposed to effective electric field simultaneously. Instead, generally only the medium between the electrodes is exposed to effective electric field.

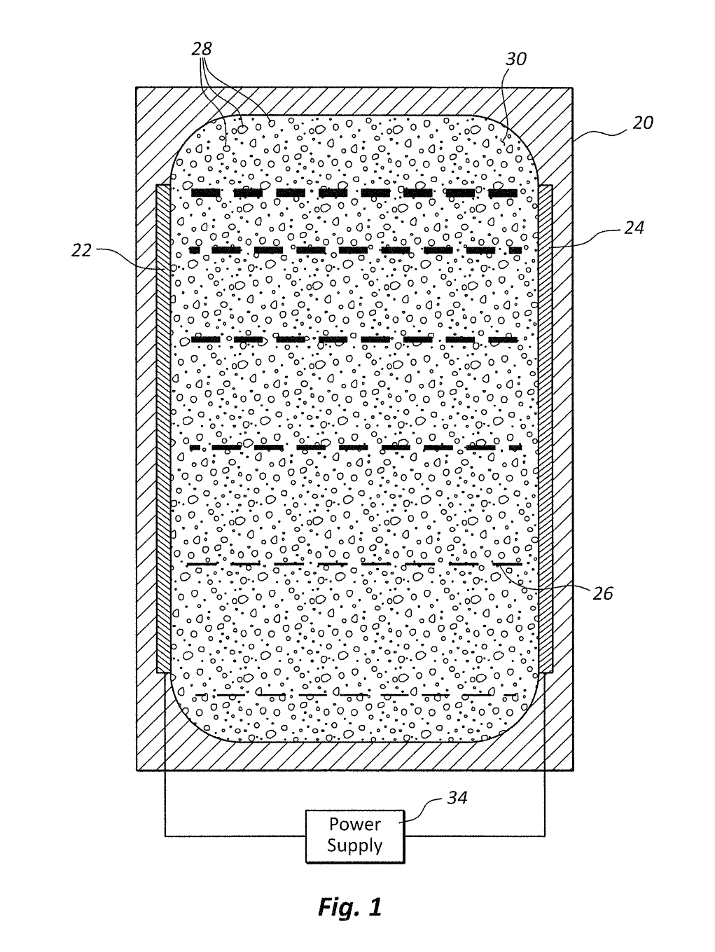

An illustration of a whole container electrode pair is shown in FIG. 1 as a vertical cross section. The outer lines represent the walls of the container 20, such as a tank, which define an interior volume 30. The container 20 can generally be used to retain the aqueous medium 26 containing the biomass feedstock. The container 20 can include various types of tanks, tubes, conduits, circular tanks, a raceway, or other suitable device configured to retain a liquid medium, including known and future developed devices. Yet, in other embodiments, the liquid medium is retained naturally, such as in a pond or other non-container environment. With these embodiments, the system can be suitably modified to provide the electrodes within the liquid medium of the pond or other location.

Mounted inside two opposing tank walls are the anode 22 and cathode 24 plates respectively. Inside the container 20 and in contact with both the anode 22 and cathode 24 is the aqueous medium 26 containing algae 28. The plates 22, 24 comprising the electrodes may, for example, have lengths 36 and widths 32 in a ratio of about 1.1:1 to 1.5:1, 1.5:1 to 3:1, 3:1 to 6:1, 6:1 to 10:1, 10:1 to 20:1, or greater than 20:1. The electrodes may be connected to one or more power supplies 34 that supplies power to the electrodes, as described herein.

In some embodiments, the distance between a pair of electrodes can be about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 10 cm, about 0.5 cm to about 15 cm, about 0.5 cm to about 20 cm, about 0.5 cm to about 25 cm, about 0.5 cm to about 30 cm, about 0.5 cm to about 40 cm, about 0.5 cm to about 50 cm, about 0.5 cm to about 75 cm, about 0.5 cm to about 100 cm, about 0.5 cm to about 120 cm, about 0.5 cm to about 150 cm, about 0.5 cm to about 200 cm, about 0.5 cm to about 300 cm, or greater than 300 cm. The electrode plates will usually be sufficiently thick to have sufficient mechanical strength considering the material(s) of which to plate is constructed to allow normal handling without problematic deflection of or damage to the plate. In many cases, the plate thickness will be about 0.2 to 0.5 mm, 1.0 to 2.0 mm, 2.0 to 5.0 mm, 4.0 to 10.0, or 0.2 to 4.0 mm.

The electrode plates surface area can be chosen in view of several parameters, such as, the desired total current, power supply capacity, desired fluid residence time, and/or desired processing capacity. For example, the individual electrode plates have exposed active areas of 1.0 to 5 cm², 5.0 to 10.0 cm², 10 to 50 cm², 50 to 200 cm², 200 to 1000 cm², or even more. Depending on the application (e.g., considering space available in desired location and/or for providing appropriate residence time for medium flowing through the electrode set), different shapes of electrode plates may be desirable, for example, commonly rectangular, which may be square or rectangular. Non-square rectangular plates may, for example, have lengths and widths in a ratio of about 1.1:1 to 1.5:1, 1.5:1 to 3:1, 3:1 to 6:1, 3:1 to 6:1, 6:1 to 10:1, 10:1 to 20:1, or greater than 20:1. Additionally, the plates can have various other shapes, such as circular, elliptical, oval, square, rectangular, the shape of another polygon, or various irregular and/or random shapes.

It has been found that some materials can have a negative effect on the growth of the biomass feedstock. For example, certain metals may have harmful effect on feedstock growth. These metals may include copper, stainless steel (as an anode), aluminum, and others. These metals may cause heavy metal absorption and or stunted growth of the biomass feedstock. Accordingly, in various embodiments, own or more electrodes comprise other conductive materials such as conductive carbon allotropes and/or non-toxic metals. Non-limiting examples of conductive carbon allotropes can include graphite, graphene, synthetic graphite, carbon fiber (iron reinforced), nano-carbon structures, and other form of deposited carbon on silicon substrates. Non-toxic metals can include platinum plated material and other non-toxic metal combinations.

In some embodiment, the electromagnetic fields generated by the electrodes can be amplified with the use of ferromagnetic and ferrimagnetic material which can include an iron ore (e.g., magnetite or lodestone), cobalt, and nickel, as well as the rare earth metals, such as gadolinium, dysprosium, neodymium, and some lanthanide rare-earth metals. These magnetic materials can be incorporated into the electrodes themselves or used within the context of the fluid flow, for example, at an inlet phase of the container 20.

In many applications, only a single electrode set will be utilized for each tank and for purpose of rotifer and or pathogen sterilization, but multiple electrode sets for each tank can be used instead. Each electrode set may be driven by a separate power supply or a plurality of electrode sets may be driven by one power supply.

The electrode set is also configured to allow flow of the medium through the space(s) between the electrodes. For electrode sets, according to some configurations, having more than two electrode plates, such flow can advantageously follow a sinuous path such that the fluid passage across the space between two adjacent electrode plates and then in a substantially anti-parallel direction between the next adjacent electrode space. In some embodiments, the set of electrodes includes a stacked set of at least two, three, four, five, six, seven, eight, nine, ten, twenty, thirty or more electrodes, with gaps between adjacent electrodes. When three or more electrodes are used, the electrodes can be configured so that the anode(s) and cathode(s) are equally spaced apart and are alternative. For example, a system with three electrodes can arrange the electrodes in series with an anode, cathode, anode configuration. Alternatively, the three electrodes can include a cathode, anode, cathode configuration. Similarly, with a six-electrode configuration, the electrodes can have an anode, cathode, anode, cathode, anode, cathode configuration. If desired, one or more non-electrode plates may be installed between successive electrode plates to serve as equipotential surfaces, thereby assisting in maintaining reasonably uniform electric fields between successive electrodes. The spacing between successive electrode plates can be chosen such that appropriate electric field strengths and/or currents are provided between the electrodes.

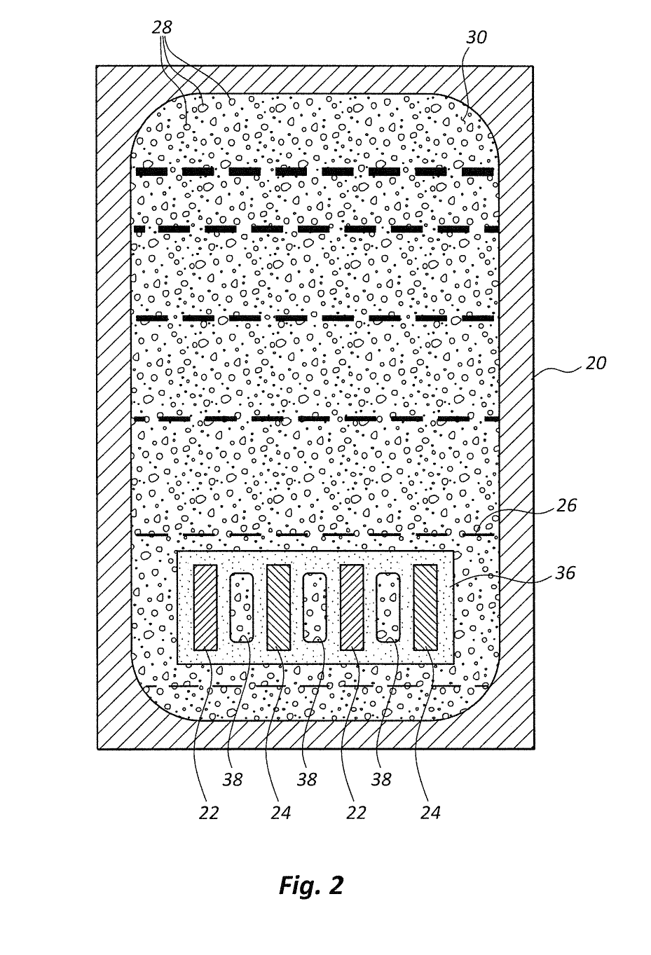

An illustrated example of such an electrode set having more than two electrode plates is depicted in FIG. 2. Other flow designs can also be implemented, for example, single pass flow across all electrode spaces in the electrode set, radial flow, and diagonal flow. In some configurations, the flow rate may be adjusted in view of the flow pattern to provide adequate residence time for the cells in the electric field for the extraction to effectively occur.

Electrode plate sets of this nature may be installed within a container 20 or in a bypass loop or transfer passageway. As depicted in FIG. 2, when installed within a container 20, the electrode set may optionally be configured as a submerged isolation electrode by substantially electrically isolating the electrode plate set from the bulk medium, for instance, by encapsulating the plate electrode set within an electrically insulating housing 36 or similar structure. Medium 26 can then pass through the electrode set by entering through an opening 38 which creates a path having high electrical resistance compared to the electrical resistance between adjacent electrodes (e.g., an inlet with cross sectional area much smaller than the area of the electrode plate and/or an outlet with cross sectional area much smaller than the area of the electrode plate and/or a current path much longer than the current path between adjacent electrodes). Similar configurations may be used in a bypass loop or transfer passageway. That is, fluid enters a sealed plate electrode set through a pipe, tubing, or other passageway for return to the tank or to another transfer destination.

In other examples, the electric field can be applied inline (e.g., when algae culture is pumped through a bypass loop or fluid transfer passageway) rather than applying electrical current in the bulk media. Anode and cathode configurations could include an inner conductive rod or tube and an outer conductive tube internally spaced equally apart which provides a fluid flow pathway between the inside wall of the outer tube and outside wall of the inner rod or tube.

The voltage (creating the electric field with resulting electric current) is applied across that space. This spacing additionally provides a high voltage transfer from the inner rod or tube through the electrical medium to the outer tube. This anode and cathode configuration could allow this method to be incorporated as a medium flow conduit.

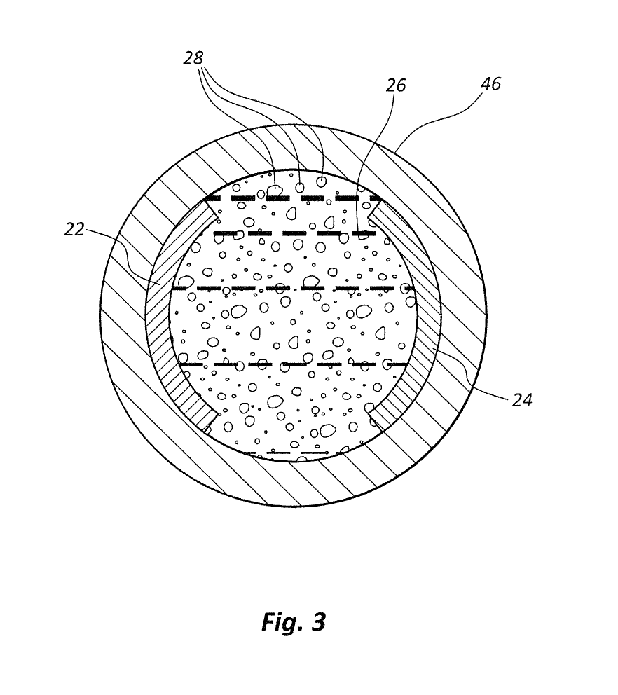

For example, FIG. 3 illustrates a bypass loop or transfer passageway having an electrode set. That is, fluid enters a sealed plate electrode set through a pipe 46, tubing, or other passageway, follows the designed flow path through the set, and exits through another passageway for return to the tank or to another transfer destination.

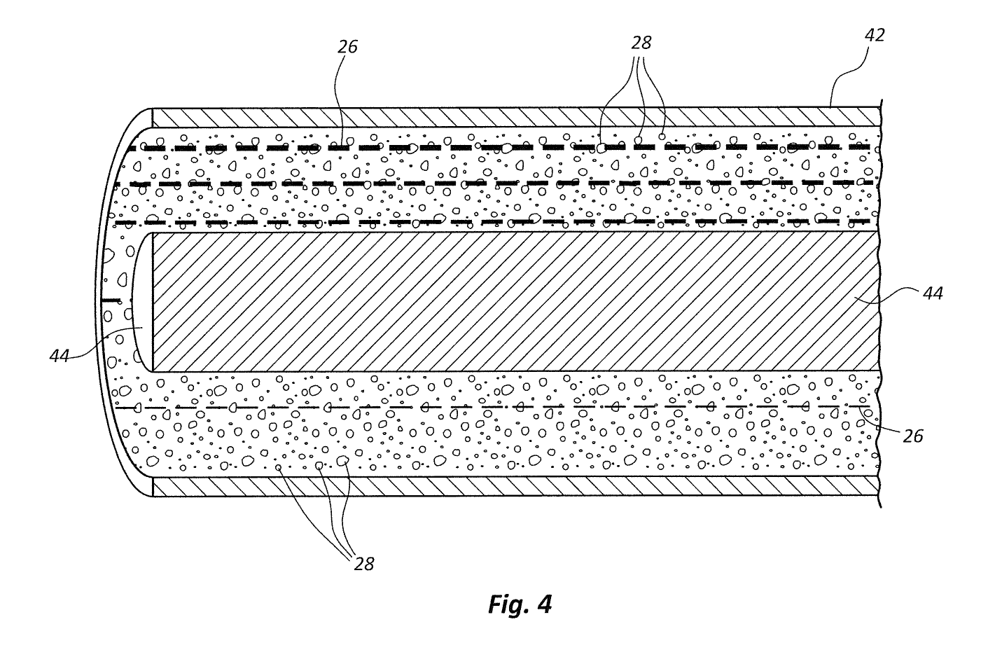

As depicted in FIG. 4, in other embodiments, the electric field can be applied inline (such as when algae culture is pumped through a bypass loop or fluid transfer passageway) rather than applying electrical current in the bulk media. Anode and cathode configurations could include an inner conductive rod 44 or tube and an outer conductive tube 42 internally spaced equally apart which provides a fluid flow pathway between the inside wall of the outer tube and outside wall of the inner rod or tube. The voltage (creating the electric field with resulting electric current) is applied across that space. This spacing additionally provides voltage transfer from the inner rod 40 or tube through the electrical medium to the outer tube 42. This anode and cathode configuration could allow this method to be incorporated as a medium flow conduit.

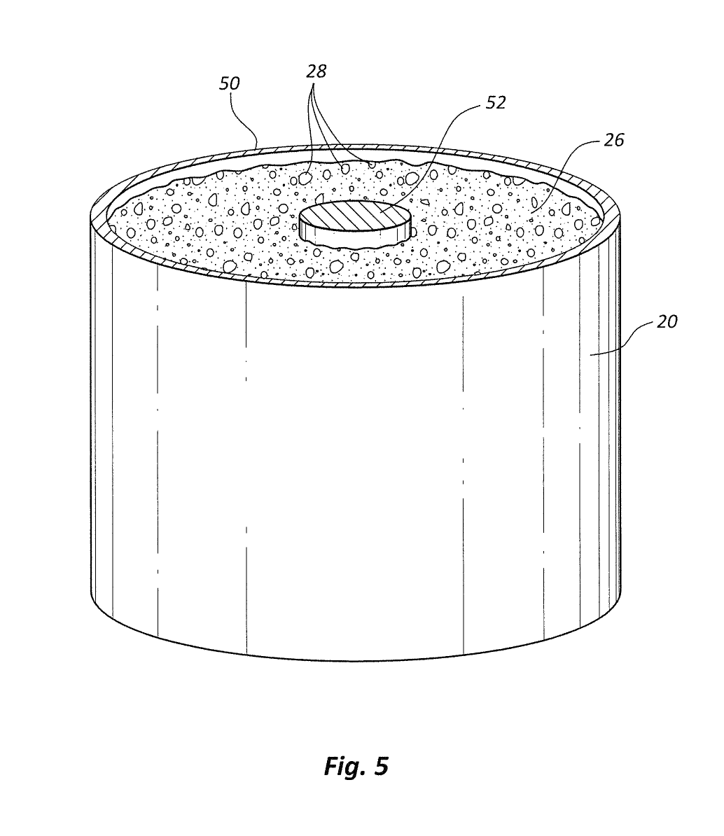

As depicted in FIG. 5, in some embodiments, the electrodes may comprise at least one whole-tank electrode. For example, as shown, a circular tanks 20 allows for the placement of a perimeter wall electrode 50 (e.g., anode) having a preferred size and thickness and a central electrode 52 (e.g., cathode). The central electrode 52 can, for example, be a cylinder such as a rod or tube located in the direct center of the tank such that the central electrode is essentially equidistant from the perimeter wall electrode 50 at all points. This practice allows a voltage to be applied substantially throughout the tank causing current to flow through the aqueous medium 26 between the electrodes 50, 52. In yet other examples, for rectangular tanks anode and cathode 24 can be installed at opposite walls inside the container 20. As in other cases, the voltage can be applied across those electrodes with resultant electric field and current flow.

Many other electrode configurations can also be utilized, all within the scope of this invention. Examples of suitable electrode configurations that can be used to flow high volumes of algae media across a high surface area electrode are described in U.S. patent application Ser. No. 12/907,024, filed Oct. 18, 2010, which is hereby incorporated herein by reference.

### Power Supply and Electric Field Modulation

For the present methods and associated systems, power supplies provide the electrical power to the electrodes causing lipid release and or remediation from invading species. Any of a variety of different types of power supplies may be chosen, for example, depending on the particular application, including, for example, electrode configuration, processing capacity, and or algal strain. In any case, the power supply should provide a desired and adequate voltage between an anode and cathode through the moderate conductivity aqueous medium. Preferred voltages, pulse shapes, and pulse frequencies can depend on the electrical conductivity of the medium and may differ for different algae species or strains and salinity.

Many different power supplies can be used for this purpose. In some cases, it may be adequate to use uninterrupted direct current (DC) power. Any of a large number of DC power supplies is available with a broad range of voltage and amperage capabilities and can be used. DC power supplies can also provide pulsed output, with pulsing capabilities being either built into the power supply or incorporated in the circuit as a separate component (s). Desirably, the output is programmable, for example, programmable voltage and/or waveform and/or pulse frequency and/or duty cycle. In many cases, a square wave output or an approximation thereof will be desirable. It is usually desirable for the power supply to be designed to handle rapidly switched loads.

An alternating current (AC) power supply can be used, with the frequency and/or voltage of the AC power selected or set at desired levels to provide effective power. The power supply can be designed to provide power at a desired voltage or the voltage can be modulated after the power supply and before the electrical power is delivered to the electrodes. As with DC power, the AC power may be supplied uninterrupted to the electrodes or may be pulsed. In many cases, it is desirable for the power to be pulsed. In such cases, preferably the power supply is designed to handle rapidly switching load.

One example of a method of providing power utilizing DC voltage, comprises of a series of coils which allows a lesser voltage input to be boosted, e.g., into kilovolt (kV) ranges. The frequency of power input to the coil is controlled by a time duration relay circuit utilized for starting and stopping electrical input to the coil. Closing the input allows the coil to electrically charge up and release the higher voltage directed to the electrodes.

The voltage frequency and the duration of time directed voltage to the primary side of the coil can be controlled utilizing pulse width modulation (PWM). If looking at a series of PWM's on an oscilloscope, sine waves appear in several different forms. For example, a peak sine wave (straight up and down) would allow shorter time duration between primary voltage inputs to the coil resulting in a lesser secondary voltage amplification. A longer duration of primary voltage can be obtained by utilizing a longer duration between the peak's drop down duration. If viewed on an oscilloscope, the result would be a plateau (square sine wave) at the top of the peak prior to the sine wave dropping back down. The result is a longer duration of primary voltage to the coil charge up time allowing for a larger amplification of voltage from the coil's secondary circuit. Further, the length duration of the square sine wave allows the kHz frequency of voltage input to the cathode.

As an example of a method utilizing AC voltage comprised a series of step up transformers which allows a lesser voltage input to be amplified into kilovolt ranges. Utilizing a capacitor inline after the transformers allows further voltage amplification due to its ability to store voltage and release this higher voltage upon reaching the capacitor's storage limits. Voltage produced is directed to the cathode. In reference to AC voltage, unless otherwise indicated, the voltage is RMS (root mean square) voltage.

As voltage produces its own PWM in the form of Hz cycles with AC always appearing on an oscilloscope in a wave form. AC can be altered by changing frequency. In many cases, the AC frequency will be normal line frequency, e.g., about 50 to 60 Hertz (Hz) but may be higher or lower. The number of cycles per second desired can relate to the density of the electrical medium within the containment tank. AC power will most often be provided having typical sine waveform, but can also be provided in other forms, e.g., square wave.

The voltage utilized can depend on a variety of factors, e.g., on the configuration of the electrodes, the electrical conductivity of the medium, the power pulse regime selected and/or the algal strain. For example, in some cases the voltage (AC or DC) can be selected from one or more of the following ranges about 0.1 to 0.5 V, 0.5 V, about 0.6 V, about 0.7 V, about 0.8 V, about 0.9 V, about 1 V, about 1.1 V, about 1.2 V, about 1.3 V, about 1.4 V, about 1.5 V, about 1.6 V, about 1.7 V, about 1.8 V, about 1.9 V, about 2 V, about 2.5 V, about 3 V, about 4 V, and about 5 V. In other cases, the voltage (AC or DC) can be selected from one or more of the following ranges about 0.1 millivolts (mV) to about 0.5 mV, 0.1 mV to about 1 mV, about 0.1 mV to about 1.05 V, about 0.1 mV to about 1.1 V, about 0.1 mV to about 1.15 volts (V), about 0.1 mV to about 1.2 V, about 0.1 mV to about 1.25 V, about 0.1 mV to about 1.3 V, about 0.1 mV to about 1.35 V, about 0.1 mV to about 1.4 V, about 0.1 mV to about 1.45 V, about 0.1 mV to about 1.5 V, about 0.1 mV to about 1.55 V, about 0.1 mV to about 1.6 V, about 0.1 mV to about 1.65 V, about 0.1 mV to about 1.7 V, about 0.1 mV to about 1.75 V, and about 0.1 mV to about 1.8 V, about 0.1 mV to about 1.85 V, about 0.1 mV to about 1.9 V, about 0.1 mV to about 1.95 V, about 0.1 mV to about 2 V, greater than about 2 V, about 0.75 V to about 1.8 V, about 0.75 V to about 1.5 V, about 0.75 V to about 1.3 V, or about 0.9 V to about 1.3 V, about 0.5 to about 15 V, about 15 V to about 75 V, about 75 to about 250 V, about 250 V to about 1000 V, about 1 kilovolts (kV) to about 2 kV or even higher voltages.

In some embodiments, a current may be used to limit the current delivered to an electrode pair. Specifically, in particular embodiments, the current can be selected from one or more of the following ranges about 1 milliampheres (mA) to about 5 mA, 1 mA to about 10 mA, about 1 mA to about 20 mA, about 1 mA to about 30 mA, about 1 mA to about 20 mA, about 1 mA to about 30 mA, about 1 mA to about 50 mA, about 1 mA to about 60 mA, about 1 mA to about 70 mA, about 1 mA to about 80 mA, about 1 mA to about 90 mA, about 1 mA to about 1 A, about 1 mA to about 1.05 A, about 1 mA to about 1.1 A, about 1 mA to about 1.15 A, about 1 mA to about 1.2 A, about 1 mA to about 1.25 A, about 1 mA to about 1.3 A, about 1 mA to about 1.35 A, about 1 mA to about 1.4 A, about 1 mA to about 1.45 A, about 1 mA to about 1.5 A, about 1 mA to about 1.55 A, about 1 mA to about 1.6 A, about 1 mA to about 1.65 A, about 1 mA to about 1.7 A, about 1 mA to about 1.75 A, and about 1 mA to about 1.8 A, about 1 mA to about 1.85 A, about 1 mA to about 1.9 A, about 1 mA to about 1.95 A, about 1 mA to about 2 A, about 1 mA to about 2.5 A, about 1 mA to about 3 A, about 1 mA to about 4 A, or greater than about 4 A.

In some instances, the power requirements of an electrode pair is relatively low, such as, for example, about 0.1 to about 0.2 watts (W). Accordingly, in some instances, a power supply 42 has relatively low power output requirements, enabling a variety of power supplies (not shown) to be used, including renewable power supplies. Non-limiting examples of power supplies useful in powering the system include solar cells, a wind turbine, a power grid, a battery, other suitable power supplies, and combinations thereof.

As indicated, in some configurations, it can be desired to provide pulsed power. To pulse power, the frequency of pulsing can be varied as can the duty cycle. In this context, the term duty cycle refers to the relative lengths of the on and off portions of each power cycle, and can be expressed, for example, as a ratio of the duration of the on portion of the cycle to the total time for the cycle, or as a ratio of the duration of the on portion of the cycle to the off portion of the cycle, or by stating the on and off durations, or by stating wither the on or off duration and the total cycle duration. Unless otherwise stated or is clear from the context, duty cycle will be stated herein as the ration of on duration to off duration for a cycle.

Accordingly, with embodiments that cycle an electromagnetic field on and off, the on-off cycle can have a duty cycle of about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.5, about 1:3, about 1:4, about 1:5, about 1:6, about 1:7, about 1:8, about 1:9, about 1:10, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 4:1, about 5:1, about 6:1, about 7:1, about 8:1, about 9:1, and about 10:1. Additionally, the duration of the duty cycle of any of the aforementioned ratios or the duration of other such occasional on-periods can include about 5 minutes, about 10 minutes, about 15 minutes, about 30 minutes, about 45 minutes, about 1 hour, or more than 1 hour. Such on-period duration of treatment time needed for effective harvest can vary depending on factors such as the algal strain and electrical stimulation conditions.

According to some configurations, it is contemplated that the electromagnetic field may be wave or frequency modulated to reduce predator populations and thus increase the biomass feedstock yields. In such embodiments, frequency may be modulate over a variety of frequency ranges, including, but not limited to, about 3 hertz (Hz) to about 30 Hz, about 30 Hz to about 300 Hz, about 300 Hz to about 3 kHz, about 3 kHz to about 30 kHz, out 30 kHz to about 300 kHz, about 300 kHz to about 3 MHz, about 3 MHz to about 30 MHz, out 30 MHz to about 300 MHz, about 300 MHz to about 3 GHz, or more than 3 GHz.

### Process Control

While it is practical to operate a growth system manually, it is more often desirable to at least partially automate the system. Thus, sensors can be located within the growth container to relay sensory feedback information on selected parameters to a control system (preferably computerized) programmed to take those culture feedback parameters and appropriately direct the system to either harvesting of the biomass or lipids, or remediation of the growth medium.

Thus, in some configurations, a computerized control system controls the initiation and termination of harvest and/or process parameters such as the voltage and/or energy level, pulse frequency, on and off times, and/or the length of each individual duty cycle. The computerized control system may regulate and adjust these controls based on biofeedback information received from various sensors of the system, such as pH sensors, oxygen reducing potential (ORP) sensors, density sensors, voltage or current sensors, conductivity factor sensors, and electrical conductivity sensors. For example, the computerized control system can increase the pulse frequencies, voltage levels, current levels, duty cycle, or frequency of voltage application or, for example, when elevated levels of foreign species are detected. Similarly, the system may lower such parameters when a lower density of foreign species is detected. In some instances, too much energy may damage or kill the algal cells along with the foreign species, and is preferably regulated based on the biofeedback.

In some embodiments, a dynamic power control module (DPC) is incorporated into the computerized control system. The DPC can be comprised of a series of sensors tied back to a main computer control unit, or central processor. This module can interface with existing industrial control systems and/or run stand-alone. The DPC can take feedback from pH, turbidity, oxygen reduction potential (ORP), conductivity, resistance, temperature, and/or other sensors as biofeedback. Using algorithms appropriate for the culture and desired product, the system calculates when the growth medium becomes infected with foreign species. The algorithms cycle may be based, for example, on the desired output from the algae, the algae species, the geographic region of the algae growth plant, and/or many other factors. Once the DPC has calculated that the culture is infested with foreign species, it will initiate a reduction sequence. The DPC will then control the power output of the one or more electrode pairs mentioned above.

The present system may be used with, or incorporated into, any of a wide variety of algal growth harvesting and extraction systems, including, but not limited to, systems such as those described in United States PCT Application No. 2010/031756; U.S. Provisional Application No. 61/373,365, and U.S. Provisional Application No. 61/356,435; and patents and applications cited therein, all of all of which are incorporated herein by reference in their entirety. Individual components, control functions and DPC may be integrated and managed by a Supervisory Control and Data Acquisition System (SCADA), as described below.

Some embodiments of the system include a controller. Various embodiments of controllers. Such a controller can be local to one or more sets or arrays of electrodes. The controller can also be remote, in which case a central controller communicates to the local system (which may include a local controller) via one or more communication links (e.g., wired or wireless link). Thus connected, the controller can be configured to control the voltage differential across the two or more electrodes, including turning the voltage on and off and adjusting the voltage.

In some embodiments, the controller is configured to adjust the voltage differential across the two or more electrodes in response to information acquired from the one or more sensors. For instance, as describe below, the controller is electronically coupled to one or more sensors, the one or more sensor being configured to detect one or more of the following pH, ORP, TDS, temperature, conductivity, salinity, chlorine, dissolved oxygen, cell density, CO₂, zeta potential, streaming current, streaming potential, and ammonia. The controller can be configured to process the information received from the one or more sensors and adjust the voltage differential across the two or more electrodes accordingly. For example, when foreign populations are detected, the controller can activate the system and initiate a voltage across one or more electrode pairs. In these embodiments, the controller may ensure regional and localized control of competing microorganisms a biomass feedstock in an effort to induce optimal growth therein.

### Controller and Sensor Systems

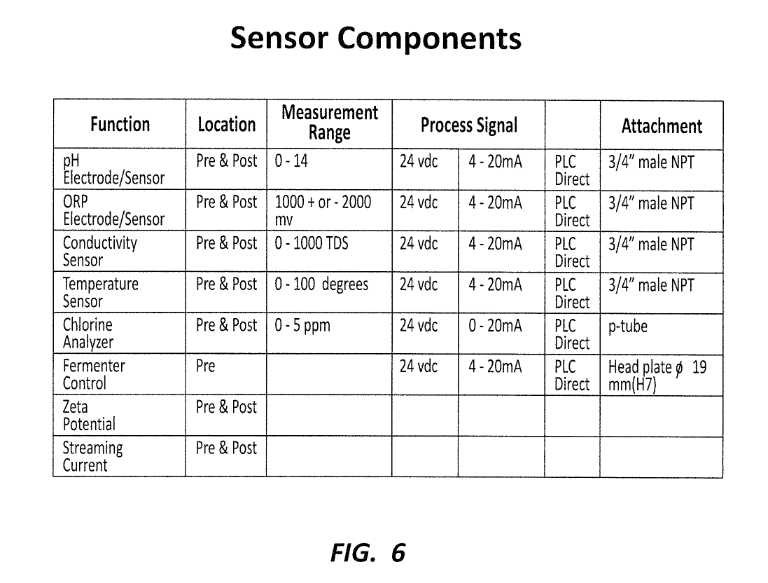

In some embodiments, the forgoing methods, systems and apparatuses involve the development and deployment of a specially selected array of sensor probes, which communicate among/between each other via Supervisory Control and Data Acquisition (SCADA) technology, and to a control module, power supplies and power conditioning units, such as pulse and frequency generators/modulators for the anode and cathode pair or zeta potential meter(s) or streaming current device(s), etc. Various sensor probes and/or related devices according to some embodiments are identified in FIG. 6 and will be discussed in greater detail below.

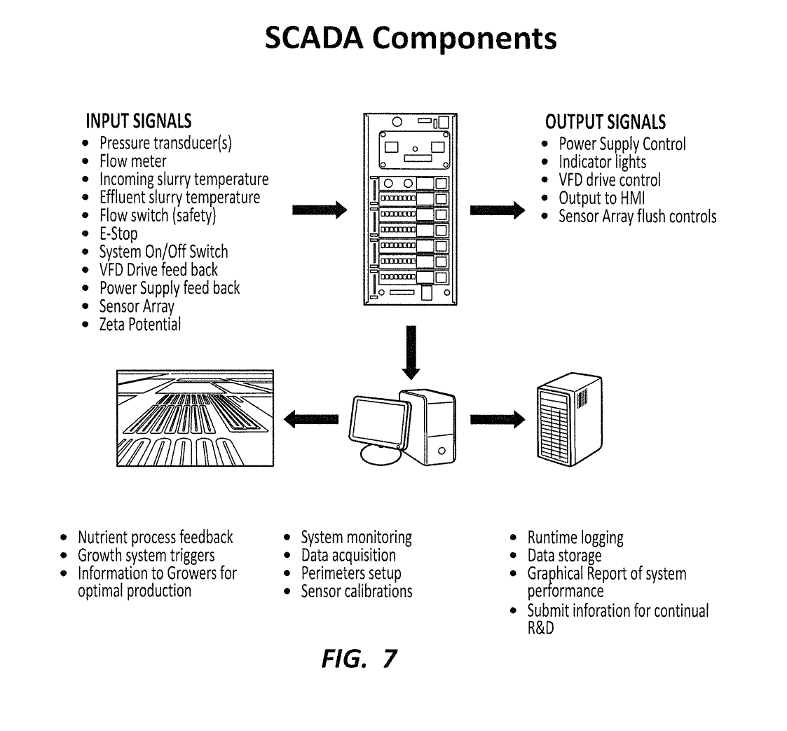

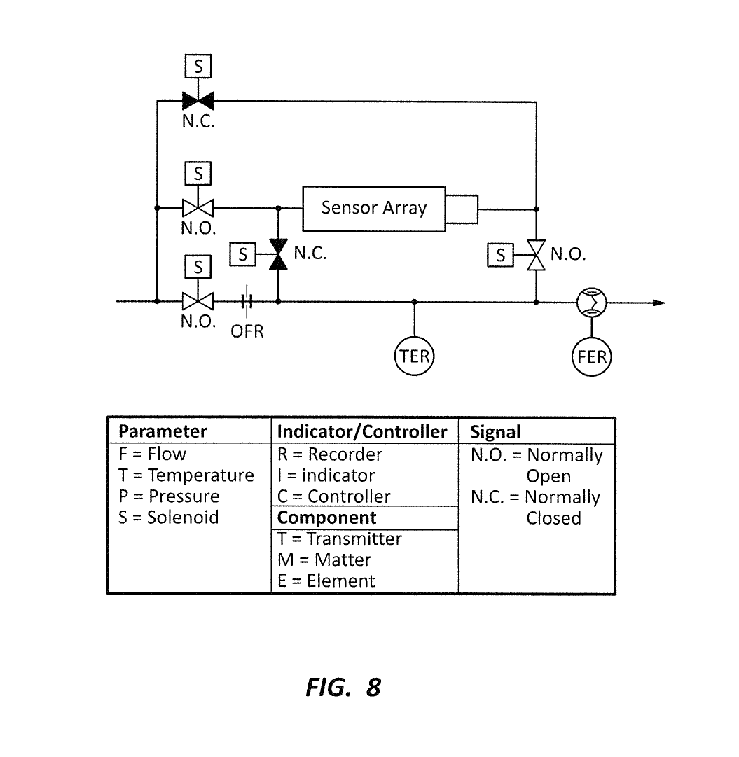

The sensor measurement parameters, according to some embodiments, are shown in FIG. 7. Some embodiments measure parameters comprising, among other things, water hardness, pH, ORP, conductivity, zeta potential, streaming current, and/or streaming potential where the dielectric properties may be quantified, and may be compared to cell density. In some embodiments, dissolved gas such as chlorine, ammonia, hydrogen, oxygen, CO₂, and other process and/or waste gas values/volumes can be used for process control and monitoring or enhancing algae growth. In yet other embodiments, additional parameters, such as temperature, are measured such that corresponding data can be used for process control. In some embodiments, process control comprises growth of algae, processing, separation and/or extraction, as well as handling the effluent, recirculation and return to process. FIG. 8 illustrates a non-limiting example of a system according to some embodiments of the invention.

An example of a sensor array according to some embodiments is shown in FIG. 9. Some embodiments of a sensor array comprise a spool piece. In some embodiments, the spool piece comprises a flow inlet. In some embodiments, the flow inlet comprises a spiraling foot. According to some embodiments, the spiraling foot may be structured to initiate a clockwise or counterclockwise flow. According to some embodiments, the clockwise or counterclockwise flow may allow the working/process fluid to move past at least one instrument probe to provide a fresh sample presentation to the instrument probe(s). In some embodiments, a series of instrument probes (e.g., at least two probes) may be staged in sequence. In some embodiments, a series of instrument probes (e.g., at least two probes) may be staged in a helix design. In some embodiments, a series of instrument probes (e.g., at least two probes) may be spaced relative to each other to resists the creation of turbulent flow within the spool.

Some embodiments may comprise a flow straightener, straightening vein, berms and/or undulations. Some embodiments may comprise a plurality of flow straighteners, straightening veins, berms or undulations. In some embodiments, at least one of the flow straightener, straightening veins, berms and/or undulations may be structured to direct flow in these directions as well.

Some embodiments comprise at least one outlet section in fluid connection to a spool piece. Some embodiments may comprise a plurality of outlet sections in fluid connection to a spool piece. Some embodiments of the outlet section comprise at least one flow restriction appliance or device. In some embodiments, the at least one flow restriction appliance may be structured to ensure the chamber of the spool can be filled with working/process fluid at all times in all positions, while flow has been established.

Some embodiments comprise elements structured to provide back flushing or chemical cleaning as shown in FIG. 9.

Some embodiments comprise central control of the dynamic flow condition inside a spool piece. Other embodiments comprise local control of the dynamic flow condition inside a spool piece. Some embodiments are structure to be used as “indication only” of the dynamic flow condition inside a spool piece. The system can also be filled with working fluid as a static/grab sampling and analytical tool, for point measurements. An example of this would be to characterize the composition of feed water in open or closed photo bioreactors, ponds or raceways in various stages of growth, maintenance, and operation. The system can also be deployed and connected to remote telemetry or local indications of water chemistry and algae culture. Lysimiter, and separate affects testing can also be accomplished remotely and unmanned, with the capability of both static and dynamic change in state scenarios. Some embodiments comprise battery operation of sensors, facilitating both local and central control. All of these configuration support data acquisition, and central signal distribution from the sending units, where instructions for set points can be modified and executed for range, and functionality, such as preset and resetting local and central alarm control, and calibration.

As mentioned above, FIG. 6 illustrates non-limiting examples of the types of sensors that may be used according to some embodiments. Sensors may be used to detect pH, ORP, TDS, temperature, conductivity, salinity, chlorine, dissolved oxygen, cell density, CO₂, zeta potential, streaming current, streaming potential and/or ammonia. An individual sensor may be used, or multiple sensors may be used. Direct probe information may be used to detect pH, ORP, TDS, temperature, conductivity, salinity, chlorine, dissolved oxygen, cell density, CO₂, zeta potential, streaming current, streaming potential and/or ammonia. In some embodiments, multiple inputs may be used to detect pH, ORP, TDS, temperature, conductivity, salinity, chlorine, dissolved oxygen, cell density, CO₂, zeta potential, streaming current, streaming potential and/or ammonia.

In some embodiments, a probe or multiple probes may be mounted via a wet-tap system to withstand a minimum of 50 psi. In some embodiments, mounting of at least one probe can be done with threaded taps and a threaded body probe and/or a compression nut system over a smooth body probe.

In some embodiments, the chemical composition of algae or other substrate may be analyzed up and/or downstream to match electrode compatibility. A higher salt content may require different electrodes and different housing than a fresh water solution.

In some embodiments, zeta potential, streaming potential and/or streaming current may be analyzed up and/or downstream to optimize processing of the algae at various stages. Some embodiments comprise a physical housing and/or sensor array structure. Some embodiments of a physical housing and/or array structure may comprise: a housing to maintain high flow while minimizing probe fouling; interior surfaces to be of a non-fouling material by means of a polished metal, ceramics or a coating that will deter formation of bio-residues; a housing to remediate air trapped and evacuation from the system; a system for bypassing the main flow for cleaning and calibrating probes; probes mounted on a helix to decrease and/or eliminate eddy currents in order to maintain a virgin sampling medium; and an array to be mounted upstream and downstream within the system.

Some embodiments comprise measurements, triggers and/or algorithmic maps for SCADA applications. Some embodiments comprise sensors connected to a SCADA system. Some embodiments comprise sensors connected to a SCADA system structured to modify the system's voltage, amperage, pulse frequency, amplitude and/or flow rates for optimal growth and/or flocculation based on a predefined series of process maps. Some embodiments comprise algorithms designed to measure point-to-point changes between upstream and post system process. Some embodiments comprise at least one probe utilized for taking at least one point measurement. Some embodiments comprise several probes structured to take at least one point measurement. Some embodiments comprise probes structured to take point-to-point comparisons (e.g., ORP).

Some embodiments comprise real time reading of probes with sampling taken on point of change. Some embodiments comprise exporting information on time intervals. Some embodiments comprise exporting information with a “dead-band” of a +/− percentage to ignore extra data points in the case of small fluctuations from system noise, wiring, air bubbles in system, etc. Some embodiments comprise probe averaging for fast response probes on 50 or 100 ms intervals. For example, some embodiments comprise probes, which read conductivity every 5 ms. As a non-limiting example, if an average of 100 ms is used, a total of 20 sample points may be averaged into a single data point. This reduces database size and false readings. Some embodiments comprise separate algorithms, which run parallel to the system process, designed to predict probe changes and monitor for irregular probe behavior. Some embodiments comprise a fault flag set via software/hardware to alert an operator to evaluate, correct and clear the fault. Some embodiments are structured to stream all information to a database server for evaluation and graphical presentation.

Some embodiments comprise systems structured for cleaning and/or antifouling. Some embodiments comprise a point injection system for cleaning of probes (e.g., argon or CO₂ blasts on point spots). Some embodiments comprise a manifold structured with a bypass valve for servicing and/or cleaning. Some embodiments comprise, in conjunction with a bypass loop, a system of valves structured, when actuated, to reverse the process flow through the spool backwashing areas of biomass/particulate build up. In some embodiments, all probes/and or some of the probes will still function properly during the backwash phase. In some embodiments, downstream probes will tend to foul faster than upstream ones, therefore special care may be utilized for cleaning. In some embodiments, alternatives to water/chemical/gas cleaning may be utilized. For example, ultrasonic emissions during a high pressure rinse may be utilized with some embodiments. Further, some embodiments may utilize a chemical that would emulsify, via ultrasonics, oils, and contaminants to assist in the cleaning of probes.

As mentioned above, FIG. 7 illustrates SCADA components, which may be utilized individually or in combination with each other according to some embodiments. Some embodiments comprise growth systems which may be structured to utilize nutrient process feedback, growth system triggers and information to growers for optimal production. For example, zeta potential can be utilized in with some embodiments. Some embodiments comprise an HMI display(s) comprising: system monitoring, data acquisition, perimeters setup and/or sensor calibrations. Some embodiments comprise database(s) comprising runtime logging, data storage, graphical report of system performance and/or additional information to be used for research and development.

FIG. 8 illustrates a non-limiting example of a system according to some embodiments. FIG. 8 is shown with the valves in normal operation. By changing the valve positions, flow may be redirected to the outlet side of the spool, thus flowing backward causing particle build up from the “normal” direction to be flushed out while still passing fluid over the sensor array. Flushing periods may be based on speed of medium passed through the spool and types of particulates in solution.

Some embodiments comprise at least one OFR (Orifice Flow Restrictor) which may be structured to divert 15 to 30 percent of the medium to the spool for sampling. Some embodiments comprise at least one flow meter attached to the end of the system that is used in conjunction with the flocculation system to control pump output and log process volumes. Some embodiments comprise at least one sensor array which is a proprietary spool type sensor array installed either upstream, downstream or on both sides of the flocculation equipment for monitoring and calibration of the power and flow delivery. In some embodiments, installation may be in a no-turbulent zone of the process. In some embodiments, locations may be a minimum of 12″ away from any bend or piping restriction and at least 24″ to 36″ from any pump. In some embodiments, sensors may be mounted vertically or horizontal in such a fashion to not allow air to get trapped in the system. Some embodiments are structured to have a 20 GPM max flow rate. Some embodiments are structured to not exceed 50 PSI.

As illustrated in FIG. 9, some embodiments comprise wiring specifications comprising: a 4 to 8 sensor array; a 4-20 ma output signal-self powered (powered from sensor); cabling from a sensor junction into a common NEMA 4X PVC enclosure with a single12pin connection (Amphenol #PTOOE-14-12P or equal) with an individual shield 18awg-6pr trunk cable back to the SCADA system; a backwash system incorporated across the spool utilizing the process fluid in a reverse direction at predefined time intervals based on fluid speed (e.g., a slower flow rate allows deposits to accumulate faster, thus a more frequent flushing interval is required); and a valve sequencing controlled by a PLC system.

The SCADA system previously described with reference to FIGS. 6 through 9 is, according to some embodiments, adapted to facilitate identifying, measuring and controlling key parameters in relation to other biomass developing processes and bio-refining processes so as to maximize the efficiency and efficacy of such processes while standardizing the underlying parameters to facilitate and enhance large-scale production of bio-based products and/or bio-energy.

### EXAMPLES

### Example 1

In a first test, an anode plate and cathode plate were placed inside a holding tank along the sides of the tank. The tank contained a living algae biomass, of a healthy Nannochloropsis culture. A control sample was taken prior to the test and was examine under a microscope set on a 40× magnification to determine the condition of the biomass and if possible to gauge the number of rotifers present in the droplet sample. It was noted, the biomass appeared to be in good condition and approximately ten rotifers were identified and counted within a contained one square centimeter area.

During this first test, a power supply applied an electric voltage across the electrode pair. A voltage differential of 1 V was applies, with a measured current of 5 mA. This voltage was applied to the holding tank for approximately five minutes. After the five minutes, a biomass sample was taken and inspected, as before. In viewing the sample it was noted that the algae biomass appeared to be in stable condition. No biomass flocculation was observed and the number of rotifers observed was only to six within a contained one square centimeter area. Thus, the number of rotifers decreased by approximately 40%.

### Example 2

In a second test, the first test was repeated using a new biomass sample and different power characteristic. This biomass also appeared to be in good condition and had approximately eight rotifers within a contained one square centimeter area. In this test, a voltage of 1.3 V was applied, with a measured current of 8 mA. This voltage was applied to the holding tank for approximately five minutes. After the five minutes, a biomass sample was taken and inspected. Upon microscope inspection it was noted again the biomass remained stable, having no detected flocculation. In this instance, the number of rotifers were once again reduced down from eight to five. The remaining five were observed to still retain movement capabilities.

In light of the first and second tests, it was observed that rotifers can be killed using an electric field, while preserving stable biomass. It is believed that further exposure can further reduce rotifer populations, while not harming the biomass feedstocks.

### Example 3

A third test was conducted using another new biomass sample, using the same techniques as in the first two tests, but with a voltage of 1.75 V and a current of 1.0 A. Upon microscope inspection, after the 5 minute test exposure, biomass flocculation was evident. However, there were no rotifers present. It thus appears that these voltage and amperage settings were unhealthy for the algae biomass.

### Example 4

In view of the following mentioned lab study, as well as additional research, it was noted an algae biomass appears to have a natural voltage presents of 0.5 volts in a static state. Therefore, due to the minor amount of voltage and current required for rotifer and pathogen control, a forth experiment to determine if static voltage and current could be increased while flowing the biomass between an electrode pair. In this test, the voltage was elevated to 1.1 V and biomass was flown between the electrodes at a rate of one gallon per minute.

In order to achieve the 1.1 volts, the anode and cathode were moved closer together at a distance of approximately one inch apart in order to increase electrical transfer within the liquid. The biomass was flown between the anode and the cathode. After periodic observations of these biomass feedstock, it was observed that rotifers and pathogens began to be eliminated from the algae biomass after being a period of approximately five minutes. No damage was observed to the biomass feed stock.

### Example 5

A fifth experiment was established to determine if by flowing the living biomass between a series of electrical isolator points placed on the anode plate, which in simulated a pulsing effect, would allow a higher voltage threshold exposure level before biomass flocculation occurred. Accordingly, five one inch electrical isolating segments were blocked out on an anode plate. During the test periodic inspections were made. It was observed that that biomass flocculation was not noticeable until voltage levels exceeded a 2 V range. Previous experiments had observed flocculation in approximately the 1.7 V range. Accordingly, it was also noted that voltage isolation could be practiced on either the anode or cathode plate without varying the voltage exposure result.

Based on the results of these tests, it was noted that the application of an electric field to a biomass feed stock did affect both single and multi-cellular organisms based on voltage and amp current exposure time. It was also noted that voltage isolation can be practiced on either the anode or cathode plate without varying the voltage exposure result.

In light of the foregoing, it will be understood that the use of low powered electrical stimulation can be used to target and destroy invasive organisms such as competing algal species, harmful bacteria, or predatory zooplankton such as rotifers.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for inhibiting the growth of competing microorganisms in a growth medium, comprising exposing the growth medium to an electric field sufficient to kill competing microorganisms and insufficient to cause flocculation to an algae population.

2. The method of claim 1, wherein growth medium to an electric field includes introducing the growth medium between two or more electrodes having a voltage of at least 0.5 V applied across the electrodes.

3. The method of claim 2, wherein exposing the cells to an electric field includes introducing the cells between two or more electrodes having a voltage differential therebetween selected from a group consisting of about 0.5 V, about 0.6 V, about 0.7 V, about 0.8 V, about 0.9 V, about 1 V, about 1.1 V, about 1.2 V, about 1.3 V, about 1.4 V, about 1.5 V, about 1.6 V, about 1.7 V, about 1.8 V, about 1.9 V, about 2 V, about 2.5 V, about 3 V, about 4 V, and about 5 V applied across the electrodes.

4. The method of claim 1, wherein the electric field is a direct current field.

5. The method of claim 1, wherein the electric filed is an alternating current field.

6. The method of claim 1, wherein the electric field is pulsed.

7. The method of claim 1, wherein the electric field is pulsed with a frequency of at least 1 Hz.

8. The method of claim 7, wherein the electric field is pulsed with a frequency selected from a group comprising 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.

9. A system for inhibiting the growth of competing microorganisms in a growth medium, the system comprising at least two electrodes connected with an electrical power supply and configured to apply an electric field across a growth medium of sufficient magnitude to kill competing microorganisms and of insufficient magnitude to cause flocculation to an algae population.

10. The system of claim 9, the system further comprising a container having a liquid capacity of at least 10 liters.

11. The system of claim 10, where the liquid capacity of the container is selected from a group consisting of about 12 liters, about 15 liters, about 20 liters, about 30 liters, about 50 liters, about 60 liters, about 75 liters, about 100 liters, about 150 liters, about 200 liters, about 250 liters, about 300 liters, about 400 liters, about 500 liters, about 750 liters, about 1,000 liters, about 2,000 liters, about 5,000 liters, and about 10,000 liters.

12. The system of claim 10, wherein the at least two electrodes are mounted in a fluid bypass loop or fluid transfer passageway fluidly connected with the container.

13. The system of claim 9, wherein the at least two electrodes comprises a stacked set of at least three electrodes with gaps between adjacent electrodes.

14. The system of claim 9, wherein the at least two electrodes are concentrically arranged.

15. The system of claim 9, wherein the power supply electrically connected with the at least two electrodes applies a voltage across the electrodes of at least 0.5 V.

16. The system of claim 15, wherein the power supply electrically connected with the electrodes applies a voltage across the at least two electrodes selected from a group consisting of about 0.5 V, about 0.6 V, about 0.7 V, about 0.8 V, about 0.9 V, about 1 V, about 1.1 V, about 1.2 V, about 1.3 V, about 1.4 V, about 1.5 V, about 1.6 V, about 1.7 V, about 1.8 V, about 1.9 V, about 2 V, about 2.5 V, about 3 V, about 4 V, and about 5 V applied across the electrodes.

17. The system of claim 9, wherein the electric field is a pulsed electric field.

18. The system of claim 17, wherein the electrical power is pulsed at a frequency of at least 1 Hz.

19. The system of claim 18, wherein the electrical power is pulsed at a frequency selected from a group comprising 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.

20. A system for inhibiting the growth of competing microorganisms in a growth medium, the system comprising:
two or more electrodes connected with an electrical power supply and configured to apply an electric field across a growth medium of sufficient magnitude to kill competing microorganisms and of insufficient magnitude to cause flocculation in an algae population;
one or more sensors configured to sense biofeedback data from the growth medium; and
a computerized control system in electronic communication with the one or more sensors, the computerized control system adjusting the parameters of the electric field based on the biofeedback data received from the one or more sensors.

21. The system of claim 20, wherein the parameters of the electric field include at least one of voltage levels applied between the two electrodes, pulse frequency, and duty cycle on and off times.

22. The system of claim 20, wherein the one or more sensors are in fluid communication with the growth medium.

23. The system of claim 20, wherein the one or more sensor are selected from a group consisting of a pH sensor, an oxygen reducing potential (ORP) sensor, a density sensor, a voltage sensor, a current sensor, a conductivity factor sensor, an electrical conductivity sensor, and combinations thereof.

24. The system of claim 20, wherein the power supply electrically connected with the electrodes applies a voltage across the two or more electrodes of at least 0.5 V.

25. The system of claim 24, wherein the power supply electrically connected with the two or more electrodes applies a voltage across the electrodes selected from a group consisting of about 0.5 V, about 0.6 V, about 0.7 V, about 0.8 V, about 0.9 V, about 1 V, about 1.1 V, about 1.2 V, about 1.3 V, about 1.4 V, about 1.5 V, about 1.6 V, about 1.7 V, about 1.8 V, about 1.9 V, about 2 V, about 2.5 V, about 3 V, about 4 V, and about 5 V applied across the electrodes.

26. The system of claim 20, wherein the electric field is a pulsed electric field.

27. The system of claim 26, wherein the electrical power is pulsed at a frequency of at least 1 Hz.

28. The system of claim 27, wherein the electrical power is pulsed at a frequency selected from a group consisting of 1 Hz, 2 Hz, 3 Hz, 5 Hz, 10 Hz, 15 Hz, 20 Hz, 30 Hz, 40 Hz, 50 Hz, 75 Hz, 100 Hz, 250 Hz, 500 Hz, 1 kHz, 2 k Hz, 5 kHz, 10 k Hz, 20 kHz, 30 Hz, and 50 kHz.
